(19) United States
(12) Patent Application Publication
(10) Pub. No.: US 2024/0173255 A1
(43) Pub. Date: May 30, 2024

(54) Polymeric Micelle Coated with Chain-Like Poorly Soluble Drug, Preparation Method and Application

(71) Applicant: Hangzhou Hanjing Biotechnology Co., Ltd, Hangzhou (CN)

(72) Inventors: Benfang Ruan, Hangzhou (CN); Jinzhang Fang, Hangzhou (CN)

(73) Assignee: Hangzhou Hanjing Biotechnology Co., Ltd, Hangzhou (CN)

(21) Appl. No.: 18/286,843

(22) PCT Filed: Apr. 13, 2022
(86) PCT No.: PCT/CN2022/086620

§ 371 Date: Oct. 13, 2023

(30) Foreign Application Priority Data
Apr. 13, 2021 (CN) 202110396162.1

(51) Int. Cl. A61K 9/107; A61K 31/41; A61K 31/501; A61K 47/34; A61K 47/60

(52) U.S. Cl. CPC A61K 9/1075; A61K 31/41; A61K 31/501; A61K 47/34; A61K 47/60

(57) ABSTRACT

Provided are a poorly soluble chain-like drug-entrapped polymeric micelle, and a preparation method and use thereof. In the polymeric micelle, a chain-like selenium-containing glutaminase inhibitor is entrapped by a carrier of polyvinylcaprolactam-polyvinyl acetate-polyethylene glycol. The prepared poorly soluble chain-like drug-entrapped polymeric micelle could effectively increase a solubility of the chain-like selenium-containing glutaminase inhibitor, and could also improve stability of the selenium-containing glutaminase inhibitor in blood and liver microsomes, thereby enhancing a bioavailability of the chain-like selenium-containing glutaminase inhibitor.

### CROSS REFERENCE TO RELATED APPLICATION

This application is a national stage application of International Patent Application No. PCT/CN2022/086620, filed on Apr. 13, 2022, which claims priority of the Chinese Patent Application No. 202110396162.1, filed with the China National Intellectual Property Administration (CNIPA) on Apr. 13, 2021. The disclosure of the two patent applications is incorporated by references in their entireties.

### TECHNICAL FIELD

The present disclosure relates to the technical field of pharmaceutical preparations, and in particular to a poorly soluble chain-like drug-entrapped polymeric micelle, and a preparation method and use thereof.

### BACKGROUND

Glutamine metabolism inhibitors are a class of small molecules that mainly work by inhibiting key enzymes in tumor metabolism. In recent years, glutamine metabolism inhibitors such as bis-2-(5-phenylacetmido-1,2,4-thiadiazol-2-yl)ethyl sulfide (BPTES), Telaglenastat (CB-839), and hexane selenoline derivatives have attracted much attention. Selenium-containing glutaminase inhibitor is an active molecule that shows a targeted inhibitory effect on tumor cells, and has compound structures of selenoline, selenodiazole, and thiadiazole. For example, there are chain-like symmetrical diselenoline molecules (series A), chain-like asymmetric diselenoline molecules (series B), m-benzene asymmetric diselenoline molecules (series C), chain-like monoselenoline molecules (series D), and chain-like selenadiazole or thiadiazole molecule (series E). The series A to E have the following chemical structural formulas:

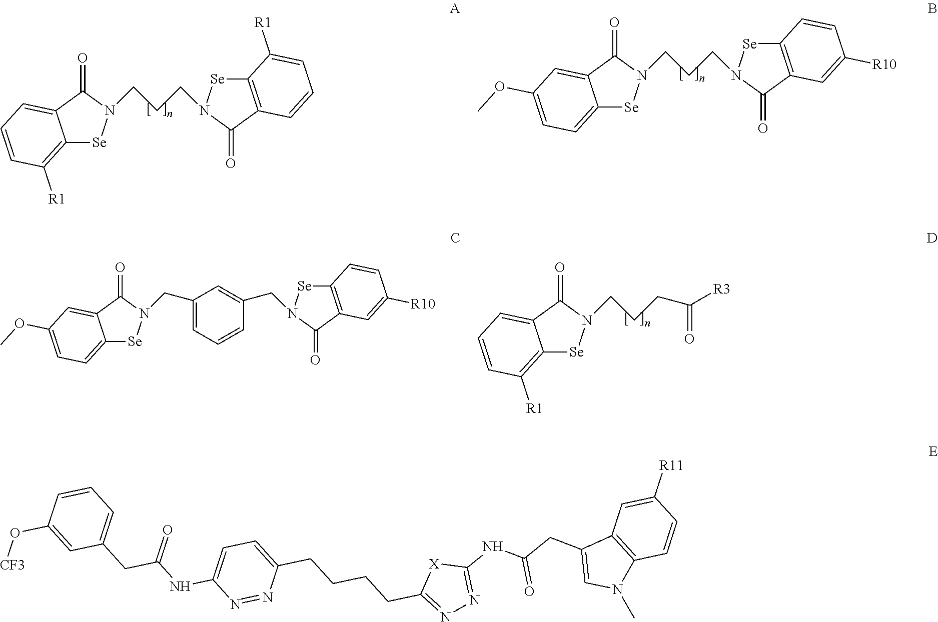

The selenium-containing glutaminase inhibitors of the series A to E have multiple effects such as anti-tumor, antioxidant, and anti-microbial effects, and show low toxic and side reactions, thereby exhibiting broad application prospects as drugs. At present, the most important research on selenium-containing glutaminase inhibitors is the development of anti-cancer preparations.

However, many glutaminase inhibitors have flexible structures and are difficult to dissolve in various solvents, including water and some organic solvents. The solubility bottleneck limits further studies on the bioactivity of the glutaminase inhibitors. For example, asymmetric hexane selenoline of the series B and methoxyselenadiazole of the series E have a water solubility of 3.3×10⁻³ g/L and 1.4×10⁻⁴ g/L, respectively. As active ingredients, the above two drugs may cause obstacles to the absorption of ordinary oral administration, and are metabolized quickly in the blood and liver microsomes, resulting in an extremely reduced amount of drug entering the blood circulation. Therefore, under injection administration, these compounds have low bioavailability and poor stability, are unstable in general dissolution systems, and are prone to precipitation and degradation generally. As a result, there is a need for preparation of suitable drug delivery systems to solve the above problems and ensure an effective improvement on the water solubility of these compounds. Further, it is also necessary to prevent active molecules in the drug from inactivation by hydrolyzation and oxidization after entering the organism, and to ensure that the active molecules are not eliminated in large quantities before reaching the tumor sites.

### SUMMARY

In order to overcome the defects and shortcomings in the prior art, an object of the present disclosure is to provide a poorly soluble chain-like drug-entrapped polymeric micelle, and a preparation method and use thereof. In the present disclosure, in the polymeric micelle, polyvinylcaprolactam-polyvinyl acetate-polyethylene glycol (trade name: Soluplus) is used as a coating carrier, which can effectively increase a solubility of a chain-like selenium-containing glutaminase inhibitor and improve a bioavailability of the chain-like selenium-containing glutaminase inhibitor. At the same time, the polymeric micelle entrapped in the chain-like selenium- containing glutaminase inhibitor can enhance a tumor treatment effect.

To achieve the above object, the present disclosure provides the following technical solutions:

The present disclosure provides a poorly soluble chain-like drug-entrapped polymeric micelle including a poorly soluble chain-like drug and Soluplus. In some embodiments, the poorly soluble chain-like drug-entrapped polymeric micelle is prepared by a film dispersion method, in which the Soluplus is used as a carrier, and the poorly soluble chain-like drug is entrapped by hydrophobic cores of polyvinylcaprolactam and polyvinyl acetate of the carrier.

In some embodiments, in the poorly soluble chain-like drug-entrapped polymeric micelle, a mass ratio of the poorly soluble chain-like drug to the Soluplus is in a range of 1:(5-100).

In some embodiments, in the poorly soluble chain-like drug-entrapped polymeric micelle, the poorly soluble chain-like drug is a selenium-containing glutaminase inhibitor, and preferably has a structure as represented by one selected from the group consisting of:

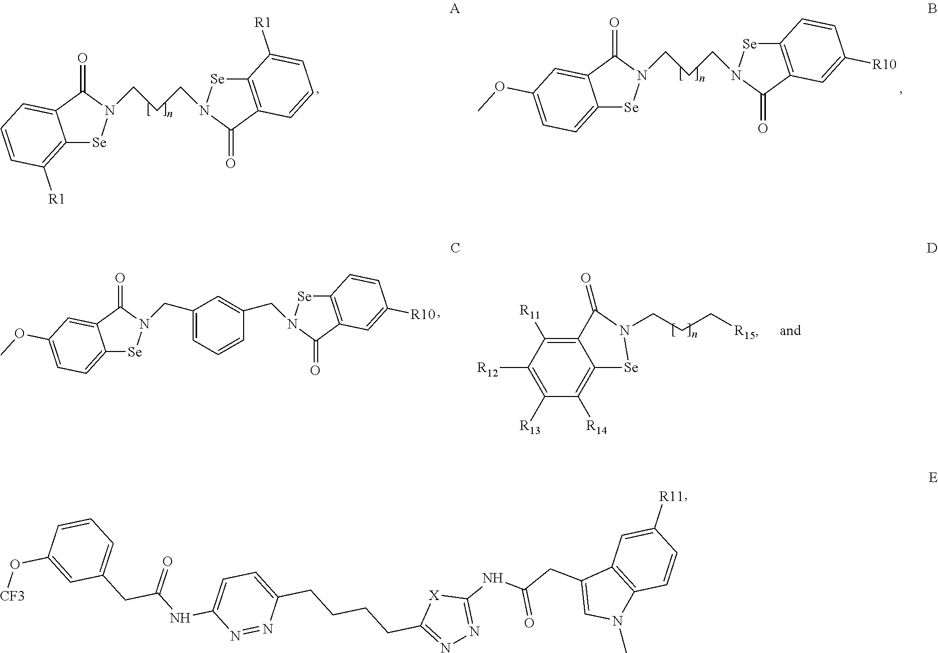

wherein, n is 1 to 4;

R₁ to R₈ are independently selected from the group consisting of H and F;

R₉ has a structure as represented by one selected from the group consisting of:

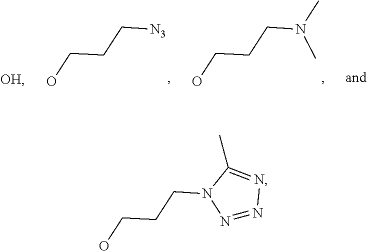

R₁₀ has a structure as represented by one selected from the group consisting of:

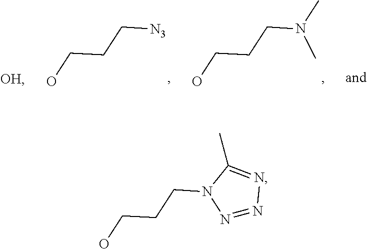

R₁₁ to R₁₄ are independently selected from the group consisting of H and F;

R₁₅ has a structure as represented by one selected from the group consisting of:

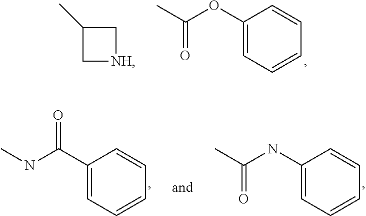

R₁₆ is selected from the group consisting of S and Se; and

R₁₇ is selected from the group consisting of H and OCH₃.

The present disclosure further provides a method for preparing the poorly soluble chain-like drug-entrapped polymeric micelle, including the following steps:

(1) dissolving the poorly soluble chain-like drug and the Soluplus in an organic solvent to obtain a mixed solution;

(2) subjecting the mixed solution obtained in step (1) to rotary evaporation to remove the organic solvent, to obtain a film, where the poorly soluble chain-like drug is entrapped by the Soluplus in the film; and

(3) subjecting the film obtained in step (2) to swelling hydration to obtain the poorly soluble chain-like drug-entrapped polymeric micelle.

In some embodiments, in the method for preparing the poorly soluble chain-like drug-entrapped polymeric micelle, the Soluplus in step (1) has a molecular weight of 90,000 to 140,000.

In some embodiments, in the method for preparing the poorly soluble chain-like drug-entrapped polymeric micelle, the organic solvent in step (1) is one or more selected from the group consisting of methanol, dichloromethane, ethanol, diethyl ether, and acetonitrile.

In some embodiments, in the method for preparing the poorly soluble chain-like drug-entrapped polymeric micelle, in step (1), a ratio of a mass of the Soluplus to a volume of the organic solvent is in a range of (0.5-100) mg: 5 mL.

In some embodiments, in the method for preparing the poorly soluble chain-like drug-entrapped polymeric micelle, a solution for the swelling hydration in step (3) is selected from the group consisting of deionized water, physiological saline, and phosphate-buffered saline (PBS); and a ratio of the solution for the swelling hydration to a mass of the Soluplus are at a volume-to-mass ratio of (2-12) mL: 200 mg.

In some embodiments, in the method for preparing the poorly soluble chain-like drug-entrapped polymeric micelle, the poorly soluble chain-like drug-entrapped polymeric micelle obtained in step (3) is subjected to a post-treatment to obtain a lyophilizer of the poorly soluble chain-like drug-entrapped polymeric micelle; and the post-treatment includes: adding a lyoprotectant into the poorly soluble chain-like drug-entrapped polymeric micelle to obtain a mixture, and subjecting the mixture to filtration, sterilization, and freeze-drying in sequence to obtain the lyophilizer of the poorly soluble chain-like drug-entrapped polymeric micelle.

In some embodiments, in the method for preparing the poorly soluble chain-like drug-entrapped polymeric micelle, the lyoprotectant is one or more selected from the group consisting of mannose, lactose, glucose, an amino acid, sucrose, and polyethylene glycol; and the lyoprotectant is used in an amount of 1% to 5% of a mass of the poorly soluble chain-like drug-entrapped polymeric micelle.

The present disclosure further provides use of the poorly soluble chain-like drug-entrapped polymeric micelle in preparation of an anti-tumor drug, an antibacterial drug, or an anti-inflammatory drug, wherein the poorly soluble chain-like drug-entrapped polymeric micelle is prepared into an oral preparation, an injection, a suppository, or a drug combination.

It can be seen from the above technical solutions that compared with the prior art, the present disclosure has the following beneficial effects:

(1) In the present disclosure, the Soluplus carrier is a multifunctional pharmaceutical adjuvant composed of three blocks: polyvinylcaprolactam, polyvinyl acetate, and polyethylene glycol. The Soluplus carrier has a molecular weight of less than 15,000 Da and shows a desirable biocompatibility.

(2) In the present disclosure, the polymeric micelle can effectively increase a solubility of the chain-like selenium-containing glutaminase inhibitor and avoid phagocytosis by the reticuloendothelial cell system in vivo. Thereby, stability of the chain-like selenium-containing glutaminase inhibitor in blood and liver microsomes is improved, while a bioavailability of the chain-like selenium-containing glutaminase inhibitor is effectively improved. Moreover, the Soluplus carrier can enhance a passive targeting ability and improve an anti-tumor effect of the chain-like selenium-containing glutaminase inhibitor.

(3) Experiments have proven that the chain-like selenium-containing glutaminase inhibitor-entrapped polymeric micelle has certain long-circulation properties in mice, indicating that the bioavailability of the chain-like selenium-containing glutaminase inhibitor is effectively improved, thereby improving a tumor inhibition effect.

### BRIEF DESCRIPTION OF THE DRAWINGS

FIGS. 1A-1H show characterization of the polymeric micelle prepared according to Example 1; where

FIG. 1A shows a color of the polymeric micelle; FIG. 1B shows a transmission electron microscopy (TEM) image of the polymeric micelle; FIG. 1C shows an average particle size of the polymeric micelle; FIG. 1D shows a potential of the polymeric micelle; FIG. lE shows a color of a Soluplus blank micelle; FIG. 1F shows a TEM image of the Soluplus blank micelle; FIG. 1G shows an average particle size of the Soluplus blank micelle; FIG. 1H shows a potential of the Soluplus blank micelle.

FIGS. 2A-2 B show stability of the polymeric micelle prepared according to Example 1; where

FIG. 2A shows an average particle size and a polydispersity index (PDI) of the polymeric micelle; FIG. 2B shows an encapsulation efficiency and a drug loading capacity of the polymeric micelle.

FIG. 3 shows a cumulative release of the polymeric micelle prepared according to Example 2 in a PBS solution.

FIG. 4 shows stability of the polymeric micelle prepared according to Example 2 in blood.

FIG. 5 shows stability of the polymeric micelle prepared according to Example 2 in liver microsomes.

FIG. 6 shows a change curve of a mouse body weight in a subcutaneous transplantation tumor test of liver cancer in H22 mice treated with the polymeric micelle prepared according to Example 2.

### DETAILED DESCRIPTION OF THE EMBODIMENTS

The technical solutions in the embodiments of the present disclosure will be clearly and completely described below. Apparently, the described embodiments are merely a part of, not all of, the embodiments of the present disclosure. All other embodiments obtained by those skilled in the art based on the embodiments of the present disclosure without creative efforts shall fall within the protection scope of the present disclosure.

The present disclosure provides a poorly soluble chain-like drug-entrapped polymeric micelle, including a poorly soluble chain-like drug and Soluplus. In some embodiments, the polymeric micelle is prepared by a film dispersion method, in which the Soluplus is used as a carrier, and the poorly soluble chain-like drug is entrapped by hydrophobic cores of polyvinylcaprolactam and polyvinyl acetate of the carrier.

In some embodiments, in the poorly soluble chain-like drug-entrapped polymeric micelle, a mass ratio of the poorly soluble chain-like drug to the Soluplus is in a range of 1:(5-100).

In some embodiments, in the poorly soluble chain-like drug-entrapped polymeric micelle, the poorly soluble chain-like drug is a selenium-containing glutaminase inhibitor, and preferably has a structure as represented by one selected from the group consisting of:

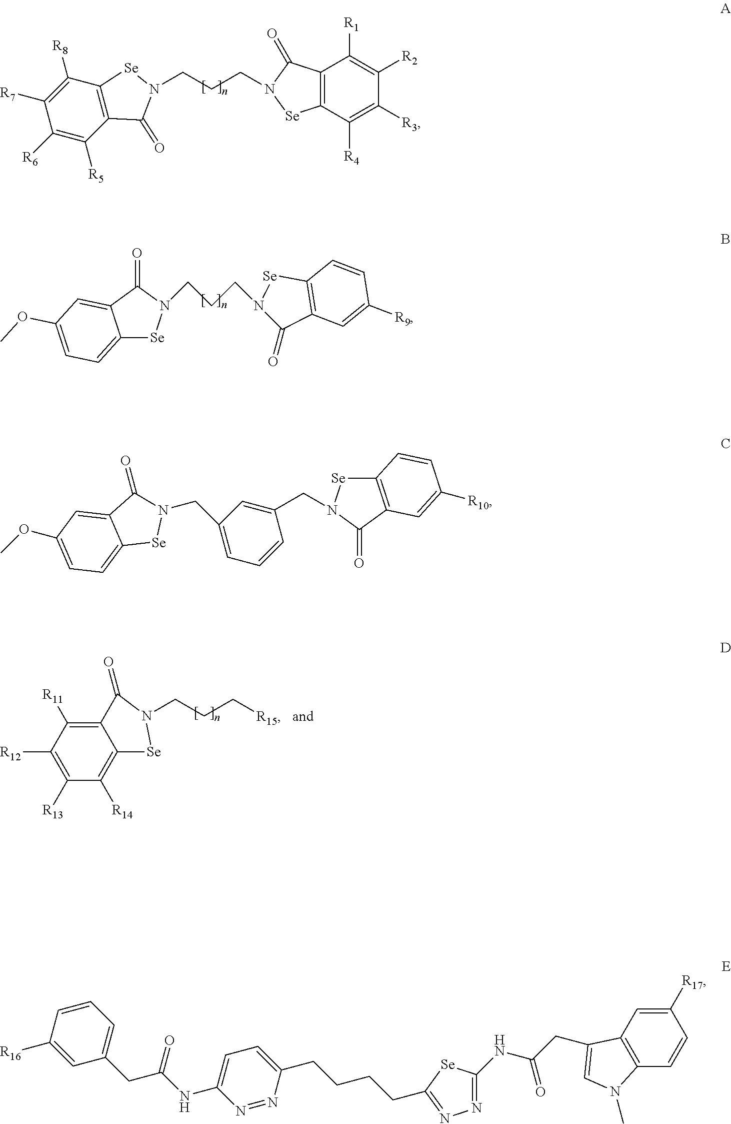

where, n is 1 to 4;

R₁ to R₈ are independently selected from the group consisting of H and F;

R₉ has a structure as represented by one selected from the group consisting of:

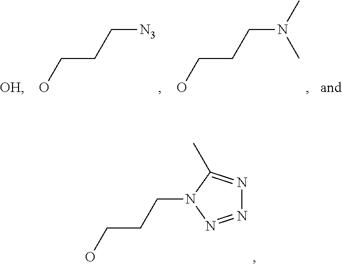

R₁₀ has a structure being one selected from the group consisting of:

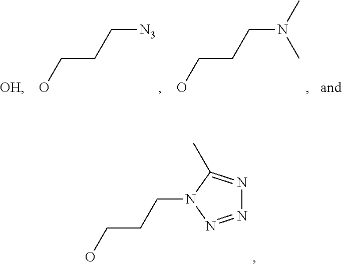

R₁₁ to R₁₄ are independently selected from the group consisting of H and F;

R₁₅ has a structure as represented by one selected from the group consisting of:

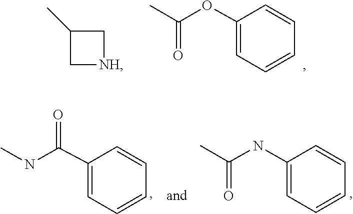

R₁₆ is selected from the group consisting of S and Se; and

R₁₇ is selected from the group consisting of H and OCH₃.

The present disclosure further provides a method for preparing the poorly soluble chain-like drug-entrapped polymeric micelle, including the following steps:

(1) dissolving the poorly soluble chain-like drug and the Soluplus in an organic solvent at a mass ratio of 1:(5-100) to obtain a mixed solution;

(2) subjecting the mixed solution obtained in step (1) to rotary evaporation at 37° C. to 45° C. to remove the organic solvent to obtain a film, where in the film the poorly soluble chain-like drug is entrapped by the Soluplus; and

(3) subjecting the film obtained in step (2) to swelling hydration at 15° C. to 40° C. to obtain the poorly soluble chain-like drug-entrapped polymeric micelle.

In some embodiments, in the method for preparing the poorly soluble chain-like drug-entrapped polymeric micelle, the Soluplus in step (1) has a molecular weight of 90,000 to 140,000.

In some embodiments, in the method for preparing the poorly soluble chain-like drug-entrapped polymeric micelle, the organic solvent in step (1) is one or more selected from the group consisting of methanol, dichloromethane, ethanol, diethyl ether, and acetonitrile.

In some embodiments, in the method for preparing the poorly soluble chain-like drug-entrapped polymeric micelle, a ratio of a mass of the Soluplus to a volume of the organic solvent in step (1) is in a range of (0.5-100) mg: 5 mL.

In some embodiments, in the method for preparing the poorly soluble chain-like drug-entrapped polymeric micelle, a solution for the swelling hydration in step (3) is selected from the group consisting of deionized water, physiological saline, and phosphate-buffered saline (PBS); and a ratio of a volume of the solution for the swelling hydration to a mass of the Soluplus is in a range of (2-12) mL: 200 mg; the PBS has a pH value of 7.2 to 7.4.

In some embodiments, in the method for preparing the poorly soluble chain-like drug-entrapped polymeric micelle, the poorly soluble chain-like drug-entrapped polymeric micelle obtained in step (3) is subjected to a post-treatment to obtain a lyophilizer of the poorly soluble chain-like drug-entrapped polymeric micelle; and the post-treatment includes: adding a lyoprotectant into the poorly soluble chain-like drug-entrapped polymeric micelle to obtain a mixture, and subjecting the mixture to filtration through a 0.22 μm microporous membrane, sterilization, pre-freezing at −80° C. overnight, and freeze-drying in a freeze dryer for 36 h to remove water in sequence to obtain the lyophilizer of the poorly soluble chain-like drug-entrapped polymeric micelle.

In some embodiments, in the method for preparing the poorly soluble chain-like drug-entrapped polymeric micelle, the lyoprotectant is one or more selected from the group consisting of mannose, lactose, glucose, an amino acid, sucrose, and polyethylene glycol; and the lyoprotectant is used in an amount of 1% to 5% of a mass of the poorly soluble chain-like drug-entrapped polymeric micelle.

The present disclosure further provides use of the poorly soluble chain-like drug-entrapped polymeric micelle in preparation of an anti-tumor drug, an antibacterial drug, or an anti-inflammatory drug, where the poorly soluble chain-like drug-entrapped polymeric micelle is prepared into an oral preparation, an injection, a suppository, or a drug combination.

Example 1

The present disclosure provided a method for preparing a poorly soluble chain-like drug-entrapped polymeric micelle, consisting of the following steps:

(1) Preparation of a poorly soluble chain-like drug: a synthesis process of a symmetric diselenoline molecule from series A was conducted as follows:

Compound CPD4 (shown in Table 2) was prepared by the following steps: 1,6-hexanediamine or an other corresponding diamine compound was added into a 50 mL flask, and then dichloromethane and TEA were added and stirred. Under an ice bath, 2-(chlorocarbonyl)-3-methylphenyl selenite or other benzene ring-substituted 2-(chlorocarbonyl)-phenyl selenite dissolved in dichloromethane was added dropwise thereto. After the addition was complete, the ice bath was removed and a resulting mixture was stirred overnight. The resulting mixture after stirring overnight was subjected to suction filtration to obtain a white solid, water and methanol were added into the white solid and stirred for 1 h. A resulting mixture was subjected to suction filtration, and dried in an oven at 50° C., obtaining the product CPD4.

MS(ESI)=479.98; LC:XDB-C184.6 mm×5 μm, methanol: water=60:40, V=1 mL/min, λ=254 nm;

¹H NMR(500 MHz, DMSO) δ7.80(dd, J=7.7,0.8Hz,1H), 7.63-7.57 (m, 1H), 7.45-7.39(m, 1H), 7.28-7.22 (m, 1H),3.71(t, J=7.1Hz,2H),1.70-1.57 (m,2H),1.35(t,J=6.8Hz,2H);

(2) 2.5 mg of the poorly soluble chain-like drug and 50 mg of a carrier material Soluplus were dissolved in 8 mL of a mixed solvent of methanol and dichloromethane, and subjected to ultrasonic treatment in a water bath for 10 min to fully dissolve the carrier material and the drug to obtain a mixed solution.

(3) The mixed solution obtained in step (2) was subjected to rotary evaporation in a rotary evaporator at 37° C. for 0.5 h, and dried under vacuum overnight to obtain a bright yellow film.

(4) The bright yellow film obtained in step (3) was subjected to swelling hydration in 5 mL of deionized water at 25° C. for 15 min, left to stand for 30 min, filtered with a 0.22 1.tm microporous membrane, and sterilized, obtaining the poorly soluble chain-like drug-entrapped polymeric micelle.

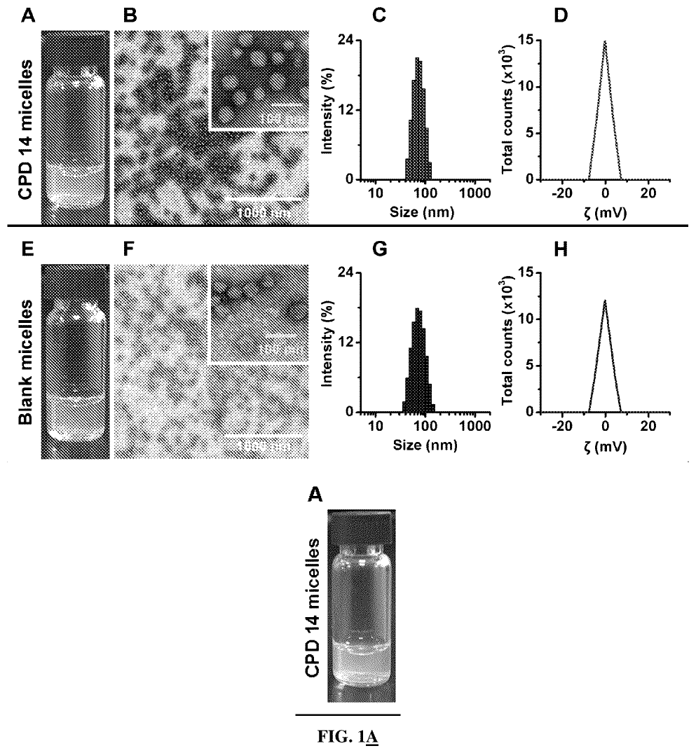

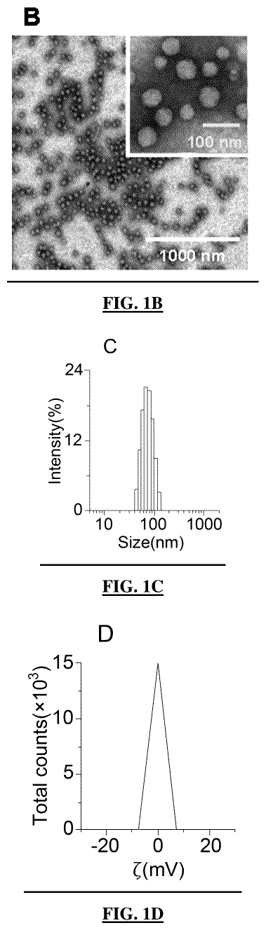

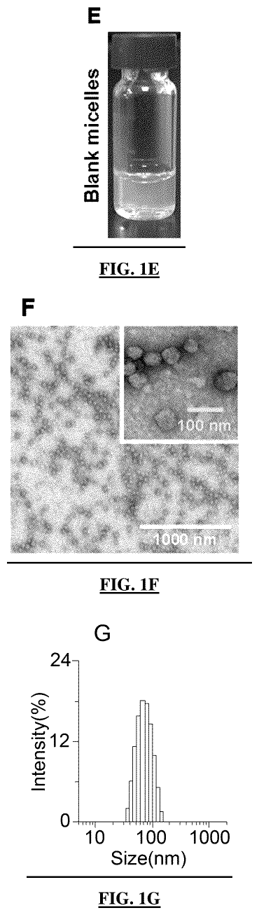

The characterization results of the polymeric micelle are shown in FIGS. 1A-1H. FIG. lA shows that the polymeric micelle of the present disclosure is a yellow transparent liquid, while FIG. lE shows that a blank micelle without drug loading is a blue liquid. According to the results measured by a Malvern laser particle size potentiometer, it can be seen that the polymeric micelle has an average particle size of about 70 nm to 80 nm (FIG. 1C) and a potential of about 0 mV (FIG. 1D); while the blank micelle has an average particle size of about 70 nm to 80 nm (FIG. 1E) and a potential of about 0 mV (FIG. 1F).

### Example 2

The present disclosure provided a method for preparing a poorly soluble chain-like drug-entrapped polymeric micelle, consisting of the following steps:

(1) Preparation of a poorly soluble chain-like drug: a synthesis process of an asymmetric diselenoline molecule from series B was conduced as follows: Compound CPD14 (shown in Table 2) was prepared by the following steps:

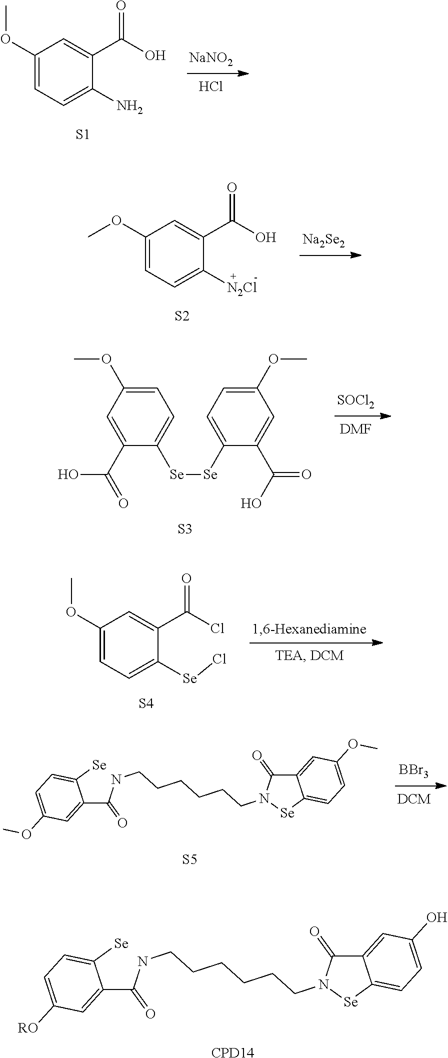

An intermediate S5 (1.07 g, 2 0 mmol) was suspended in 50 mL of dry dichloromethane, and 1M BBr3 (3.5 mL, 3.5 mmol) was added dropwise at −78° C. After the addtion was completed, a resulting reaction system was raised to ambient temperature and reacted overnight, and then added dropwise into 40 mL of a 1M HCl solution at 0° C. and stirred for 30 min, during which a precipitate was produced. The precipitate was collected by filtration, and then washed with water and diethyl ether successively. An obtained crude product was subjected to column chromatography (DCM: MeOH=50:1 to 30:1) to obtain a light yellow solid product CPD14, 534 mg, with a yield of 46%.

MS(ESI)=525.99; ¹H NMR (500 MHz,DMSO-d₆) δ9.73(s ,1H),7.90(d,J=8.5Hz,1H),7.78(d,J=8.5Hz,1H),7.30(d,J=2.5Hz,1H),7 .23(dd,J=8.5,2.5Hz,1H), 7.18(d,J=2.5Hz,1H),7.07(dd,J=8 .5,2.5Hz,1H),3.82(s ,3H),3.69(t,J=7.5Hz,2H),3.66(t,J=7.0Hz,2H),1 .64-1.54(m,4H),1.36-1.31(m,4H).

(2) 1 mg of the poorly soluble chain-like drug and 50 mg of a carrier material Soluplus were dissolved in 6 mL of a mixed solvent of acetonitrile and dichloromethane, and subjected to ultrasonic treatment in a water bath for 10 min to fully dissolve the carrier material and the drug to obtain a mixed solution.

(3) The mixed solution obtained in step (2) was subjected to rotary evaporation in a rotary evaporator at 45° C. for 0.5 h, and dried under vacuum overnight to obtain a bright yellow film.

(4) The bright yellow film obtained in step (3) was subjected to swelling hydration in 5 mL of PBS at 25° C. for 15 min, left to stand for 30 min, filtered with a 0.22 μm microporous membrane, and sterilized, obtaining the poorly soluble chain-like drug-entrapped polymeric micelle.

### Example 3

The present disclosure provided a method for preparing a poorly soluble chain-like drug-entrapped polymeric micelle, consisting of the following steps:

(1) Preparation of a poorly soluble chain-like drug: a synthesis process of an m-benzene asymmetric diselenoline molecule from series C was conducted as follows:

Compound CPD31 (shown in Table 2) was prepared by the following steps: m-xylylenediamine (272.4 mg, 2.0 mmol), triethylamine (1.54 mL, 11 mmol), and dry dichloromethane (20 mL) were added in sequence into a 50 mL round-bottom flask, and a solution of methoxyselenium chloride (1.42 g, 5 0 mmol) dissolved in dry dichloromethane (20 mL) was added dropwise thereto at 0° C. After the addition, a resulting reaction system was raised to ambient temperature and reacted overnight, during which a precipitate was produced. The precipitate was collected by filtration, and then washed with water and diethyl ether successively. An obtained crude product was subjected to column chromatography (DCM: MeOH=50:1) to obtain a light yellow solid compound m-xylylenedimethoxyselenoline, 848.7 mg, with a yield of 76%, MS (ESI)=531.95. 1M BBr₃ (3.5 mL, 3.5 mmol) was added dropwise into m-xylylenedimethoxyselenoline at -78° C. under the same conditions as those in Example 2 to obtain the product CPD31.

MS(ESI)=545 .95 ; ¹H NMR(500MHz,DMSO-d₆)δ7.86(d,J=8.5Hz,2H),7 .36-7.30(m,3H),7.29(s,1H),7.25(d,J=2.8Hz,1H),7.24-7.20(m,3H),4.88(s,4H),4.7(s,1H),3.83(s,3H);

(2) 5 mg of the poorly soluble chain-like drug and 50 mg of a carrier material Soluplus were dissolved in 6 mL of a mixed solvent of ethanol and diethyl ether, and subjected to ultrasonic treatment in a water bath for 10 min to fully dissolve the carrier material and the drug to obtain a mixed solution.

(3) The mixed solution obtained in step (2) was subjected to rotary evaporation in a rotary evaporator at 40° C. for 0.5 h, and dried under vacuum overnight to obtain a bright yellow film.

(4) The bright yellow film obtained in step (3) was subjected to swelling hydration in 5 mL of deionized water at 25° C. for 15 min, left to stand for 30 min, filtered with a 0.22 1.tm microporous membrane, and sterilized to obtain the poorly soluble chain-like drug-entrapped polymeric micelle.

### Example 4

The present disclosure provided a method for preparing a poorly soluble chain-like drug- entrapped polymeric micelle, consisting of the following steps:

(1) Preparation of a poorly soluble chain-like drug: a synthesis process of a monoselenoline molecule from series D was conducted as follows:

Compound CPD37 (shown in Table 2) was prepared by the following steps: 6-aminocaproic acid (25 mmol, 3.279 g) was suspended in 50 mL of methanol, and SOC12 (30 mmol, 3.57 g, 2.18 mL) was slowly added dropwise thereto at 0° C. A resulting mixture was refluxed for 3 h, cooled to ambient temperature, and stirred overnight. A resulting mixture obtained after stirred overnight was subjected to rotary evaporation to remove the solvent to obtain a transparent viscous liquid, which was placed in a refrigerator to obtain a white solid. 0.465 g of aniline was dissolved in 17.5 mL of dichloromethane into a 50 mL flask, and then added with 1.5 mL of TEA and stirred. 1.30 g of 2-(chlorocarbonyl)phenyl hypochlorous selenic acid was dissolved in dichloromethane and then added dropwise to the flask under an ice bath. After the dropwise addition was completed, the ice bath was removed, and a resulting mixture was stirred overnight and purified through a column to obtain the product CPD37.

MS (ESI)=402.08; 1H NMR (500 MHz,CDC13)δ8.00(d,J=8 .4Hz,1H),7.66(dd,J=8 .5,1.3Hz,2H),7 .65(d,J=8 .0Hz,1H),7 .54(dd,J=15 .2,1. 3Hz,1H),7 .50-7.42(m,3H),7 .40-7 .35(m,1H),4.74(s ,1H),3.81(t,J=7 .1Hz,2H),3.06(d,J=6.1Hz,2H),1.71 -1.63(m,2H),1.41(d,J=17 .0Hz,2H),1.33(dt,J=14.6,5 .8Hz,4H);

(2) 15 mg of the poorly soluble chain-like drug and 90 mg of a carrier material Soluplus were dissolved in 4 mL of dichloromethane, and subjected to ultrasonic treatment in a water bath for 10 min to fully dissolve the carrier material and the drug to obtain a mixed solution.

(3) The mixed solution obtained in step (2) was subjected to rotary evaporation in a rotary evaporator at 42° C. for 0.5 h, and dried under vacuum overnight to obtain a bright yellow film.

(4) The bright yellow film obtained in step (3) was subjected to swelling hydration in 5 mL of PBS at 25° C. for 15 min, left to stand for 30 min, filtered with a 0.22 μm microporous membrane, and sterilized, obtaining the poorly soluble chain-like drug-entrapped polymeric micelle.

### Example 5

The present disclosure provided a preparation method of a poorly soluble chain-like drug-entrapped polymeric micelle, consisting of the following steps:

(1) Preparation of a poorly soluble chain-like drug: a synthesis process of a selenadiazole or thiadiazole molecule from series E was conducted as follows:

Compound CPD43 (shown in Table 2) was prepared by the following steps: 1-methyl-3-indoleacetic acid (45 mg, 0.24 mol) was dissolved into 5 mL of dimethyl formamide (DMF), and then O-Benzotriazole-N,N,N′,N′-tetramethyl-uronium-hexafluorophosphate (HBTU) (91 mg, 0.24 mmol) and N,N-Diisopropylethylamine (DIPEA) (38 mg, 0 3 mmol) were added to a resulting solution in sequence. A resulting mixture was reacted at ambient temperature for 0.5 h, and then an amino-containing selenadiazole or thiadiazole intermediate (100 mg, 0 2 mmol) was added to a resulting reaction mixture, and a reaction was conducted at ambient temperature overnight. After the reaction was completed, DMF was evaporated from a resulting reaction mixture under reduced pressure, an obtained solid crude product was dissolved in ethyl acetate, washed with 10 mL of water, and transferred to a separatory funnel and subjected to liquid separation. An obtained aqueous phase was extracted twice with 20 mL of ethyl acetate, two resulting organic phases were combined, dried with anhydrous sodium sulfate, filtered, concentrated by rotary evaporation, and then separated through a column with dichloromethane: methano1=20:1 to obtain a grayish-yellow solid product CPD43 (81 mg, yield 60%).

MS(ESI)=671.12; ¹H-NMR(500 MHz, DMSO-d 6) δ12.68(s,1H),11.31(s,1H),8.19(t,J=10.0Hz,2H),7.56(dd,J=10.5,6.0Hz,1H),7.46(t,J=8.0Hz,1H),7.37( d,J=7 .5Hz,2H),7 .29(d,J=9.0Hz,1H),7 .26(d,J=8 .0Hz,1H),7 .21(s,1H),7 .10(d,J=2.5Hz,1H),6.79(dd,J=9.0,2.5Hz,1H),3.84(d,J=8.0Hz,3H),3.74(s,2H),3.71(s,2H),3.66-3.56(m,2H),3.14-3.08(m,2H),3.01-2.96(m,2H),2.90-2.84(m,2H);

(2) 6 mg of the poorly soluble chain-like drug and 70 mg of a carrier material Soluplus were dissolved in 8 mL of a mixed solvent of methanol and dichloromethane, and subjected to ultrasonic treatment in a water bath for 10 min to fully dissolve the carrier material and the drug to obtain a mixed solution.

(3) The mixed solution obtained in step (2) was subjected to rotary evaporation in a rotary evaporator at 39° C. for 1 h, and dried under vacuum overnight to obtain a bright yellow film.

(4) The bright yellow film obtained in step (3) was subjected to swelling hydration in 5 mL of physiological saline at 25° C. for 15 min, left to stand for 30 min, filtered with a 0.22 μm microporous membrane, and sterilized, obtaining the poorly soluble chain-like drug-entrapped polymeric micelle.

The average particle size, polydispersity index (PDI), and encapsulation efficiency of the polymeric micelles prepared according to Examples 1 to 5 of the present disclosure were measured, while a Soluplus blank micelle without drug entrapment was used as a control. The results are shown in Table 1. As shown in Table 1, the polymeric micelles prepared according to the present disclosure have an average particle size of 60 nm to 80 nm, a PDI of less than 0.2, and an encapsulation efficiency of not less than 94%.

TABLE 1
Average particle size, PDI, and encapsulation efficiency
of polymeric micelles and Soluplus blank micelle

| | Average particle | | Encapsulation |
|---|---|---|---|
| ID | size (nm) | PDI | efficiency (%) |
| Soluplus blank micelle | 77.7 ± 1.1 | 0.18 ± 0.02 | — |
| Example 1 | 63.4 ± 1.4 | 0.15 ± 0.003 | 95.1 ± 1.9 |
| Example 2 | 65.8 ± 4.2 | 0.14 ± 0.02 | 95.9 ± 1.6 |
| Example 3 | 64.3 ± 5.2 | 0.13 ± 0.002 | 95.6 ± 2.3 |
| Example 4 | 72.9 ± 2.1 | 0.17 ± 0.02 | 94.3 ± 6.1 |
| Example 5 | 69.5 ± 1.7 | 0.16 ± 0.002 | 96.3 ± 3.6 |

### Solubility Test:

The solubilities of the poorly soluble chain-like drugs of the series A to E according to the present disclosure in water and a 10% Soluplus solution are shown in Table 2.

TABLE 2
Solubility of poorly soluble chain-like drugs of series A to E in water and 10% Soluplus solution

| | | | | Mass | Solubility in | Solubility in |
|---|---|---|---|---|---|---|
| | | | | spectrum | water | 10% Soluplus |
| | Compound | | | (m/z; M + H⁺) | (mg/mL) | (mg/mL) |
| Series A | CPD1 | R₁═H | n = 1 | 437.92 | 0.004 | 0.2 |
| | CPD2 | R₅═H | n = 2 | 452.01 | 0.001 | 0.2 |
| | CPD3 | | n = 3 | 465.95 | 0.001 | 0.3 |
| | CPD4 | | n = 4 | 479.98 | 0.001 | 0.5 |
| | CPD5 | | n = 5 | 494.02 | 0.001 | 0.5 |
| | CPD6 | R₁═F | n = 1 | 474.01 | 0.004 | 0.02 |
| | CPD7 | R₅═F | n = 2 | 487.98 | 0.002 | 0.25 |
| | CPD8 | | n = 3 | 501.97 | 0.001 | 0.3 |
| | CPD9 | | n = 4 | 515.98 | 0.001 | 0.8 |
| | CPD10 | | n = 5 | 530.01 | 0.001 | 0.6 |
| Series B | CPD11 | R₉═OH | n = 1 | 483.95 | 0.015 | >1 |
| | CPD12 | | n = 2 | 497.98 | 0.013 | >1 |
| | CPD13 | | n = 3 | 512.01 | 0.01 | >1 |
| | CPD14 | | n = 4 | 525.99 | 0.01 | >1 |
| | CPD15 | | n = 5 | 539.89 | 0.01 | >1 |
| | CPD16 | R₉═OCH₂CH₂CH₂N₃ | n = 1 | 567.02 | <0.01 | >1 |
| | CPD17 | | n = 2 | 581.08 | <0.01 | >1 |
| | CPD18 | | n = 3 | 595.05 | <0.01 | >1 |
| | CPD19 | | n = 4 | 609.04 | <0.01 | >1 |
| | CPD20 | | n = 5 | 623.03 | <0.01 | >1 |
| | CPD21 | R₉═OCH₂CH₂CH₂N (CH₃)₂ | n = 1 | 569.07 | <0.01 | >1 |
| | CPD22 | | n = 2 | 583.04 | <0.01 | >1 |
| | CPD23 | | n = 3 | 597.05 | <0.01 | >1 |
| | CPD24 | | n = 4 | 611.08 | <0.01 | >1 |
| | CPD25 | | n = 5 | 625.03 | <0.01 | >1 |
| | CPD26 | R₉═OCH₂CH₂CH₂N₄CCH₃ | n = 1 | 608.03 | <0.01 | >1 |
| | CPD27 | | n = 2 | 622.02 | <0.01 | >1 |
| | CPD28 | | n = 3 | 636.01 | <0.01 | >1 |
| | CPD29 | | n = 4 | 650.07 | <0.01 | >1 |
| | CPD30 | | n = 5 | 664.06 | <0.01 | >1 |
| Series C | CPD31 | R₁₀═OH | Meta-position | 545.95 | <0.01 | >1 |
| | CPD32 | R₁₀═OCH₂CH₂CH₂N₃ | Meta-position | 629.02 | <0.01 | >1 |
| | CPD33 | R₁₀═OCH₂CH₂CH₂N (CH₃)₂ | Meta-position | 631.04 | <0.01 | >1 |
| | CPD34 | R₁₀═OCH₂CH₂CH₂N₄CCH₃ | Meta-position | 670.02 | <0.01 | >1 |
| Series D | CPD35 | R₁₅ = azetidine | | 324.08 | <0.01 | >1 |
| | CPD36 | R₁₅ = phenyl acid ester | | 389.05 | <0.01 | >1 |
| | CPD37 | R₁₅ = phenylamide | | 388.08 | <0.01 | >1 |
| | CPD38 | R₁₅ = benzoate amide | R₁₁═H | 388.05 | <0.01 | >1 |
| | CPD39 | R₁₅ = azetidine | | 342.07 | <0.01 | >1 |
| | CPD40 | R₁₅ = phenyl acid ester | | 407.06 | <0.01 | >1 |
| | CPD41 | R₁₅ = phenylamide | | 406.07 | <0.01 | >1 |
| | CPD42 | R₁₅ = benzoate amide | R₁₁═F | 406.06 | <0.01 | >1 |
| Series E | CPD43 | R₁₆═Se | R₁₇═H | 671.12 | 0.0003 | >4 |
| | CPD44 | | R₁₇═OCH₃ | 701.13 | 0.0003 | >4 |
| | CPD45 | R₁₆═S | R₁₇═H | 623.19 | 0.001 | >4 |
| | CPD46 | | R₁₇═OCH₃ | 653.20 | 0.001 | >4 |

Taking the symmetric diselenoline molecule CPD4 from series A prepared according to Example 1 as an example, solubilities in different solvents and carriers were measured separately. A test method was conducted as follows: 1 mg of the compound was added into 1 mL of different solvents or carriers separately, ultrasonicated in a water bath for 10 min to fully dissolve the compound to obtain a mixture, and the mixture was stirred at ambient temperature for 12 h, and centrifuged, and a compound concentration in a resulting supernatant was determined by HPLC. The results are shown in Table 3.

TABLE 3
Solubility of compound CPD4 in different solvents or carriers

| | | Solubility | Dissolution |
|---|---|---|---|
| Solvent | | (mg/mL) | multiple |
| Aqueous | Water | 0.0016 ± 0.00005 | — |
| solvent | Hydrochloric acid | 0.0022 ± 0.00026 | 1.38 |
| | (pH = 1.2) | | |
| | Sodium acetate buffer | 0.0013 ± 0.00047 | 0.81 |
| | (pH = 4.5) | | |
| | PBS | 0.0008 ± 0.00015 | 0.5 |
| | (pH = 7.4) | | |
| Hydrophilic | DMSO | 33.81 ± 0.70 | 21131 |
| solvent | DMF | 17.31 ± 0.32 | 10819 |
| | Methanol | 1.41 ± 0.13 | 881 |
| | Ethanol | 1.28 ± 0.04 | 800 |
| | Acetonitrile | 0.42 ± 0.005 | 263 |
| | Acetone | 0.44 ± 0.04 | 275 |
| | Isopropanol | 0.98 ± 0.06 | 613 |
| Hydrophobic | Ethyl acetate | 0.24 ± 0.02 | 150 |
| solvent | n-Octanol | 1.06 ± 0.08 | 662 |
| | Dichloromethane | 3.17 ± 0.16 | 1981 |
| Solid | F68 | 0.013 ± 0.003 | 8.1 |
| adjuvant | F127 | 0.098 ± 0.008 | 61 |
| | HP-β-CD | 0.060 ± 0.003 | 38 |
| | SBE-β-CD | 0.071 ± 0.004 | 44 |
| | PEG2000 | 0.007 ± 0.002 | 4.4 |
| | PEG4000 | 0.008 ± 0.001 | 5 |
| | PEG6000 | 0.006 ± 0.001 | 3.8 |
| Soluplus | Soluplus | 0.515 ± 0.008 | 400 |
| Liposome | Lecithin | 0.04 ± 0.005 | 40 |
| | Cholesterol | 0.06 ± 0.001 | 46 |

The hydrophobic constants and solubilities of the selenium-containing glutaminase inhibitors from series A to E prepared according to Examples 1 to 5 of the present disclosure as well as the commercially available ethaselen in water, dimethylsulfoxide (DMSO), and Soluplus solutions are shown in Table 4.

TABLE 4
Solubilities of ethaselen and compounds of Examples 1 to 5 in different solutions

| | | Ethaselen | Example 1 | Example 2 | Example 3 | Example 4 | Example 5 |
|---|---|---|---|---|---|---|---|
| Hydrophobic constant | | 1.87 | 2.18 | 2.56 | 3.30 | 3.57 | 2.91 |
| Water (μg/mL) | | 2-3 | 4-5 | 1-3 | 1-2 | 2-3 | 2-4 |
| DMSO (mg/mL) | | 22 | 29 | 51 | 34 | 45 | 38 |
| Soluplus | 1% | 4.7 ± 0.3 | 115 ± 7 | 93 ± 4 | 167 ± 26 | 102 ± 8 | 87 ± 5 |
| (μg/mL) | 5% | 13.9 ± 2.1 | 165 ± 6 | 116 ± 10 | 272 ± 22 | 184 ± 11 | 164 ± 15 |
| | 10% | 15.2 ± 0.8 | 232 ± 13 | 246 ± 9 | 515 ± 8 | 365 ± 10 | 389 ± 20 |
| | 20% | 48.2 ± 2.4 | 300 ± 17 | 575 ± 34 | 883 ± 25 | 512 ± 26 | 471 ± 13 |

As shown in Table 3 and Table 4, the solubilities of the selenium-containing glutaminase inhibitor prepared according to the present disclosure in the Soluplus solutions are significantly increased compared to those of other types of solutions, indicating that the polymeric micelle prepared by coating the drug with Soluplus could improve drug solubility.

### Stability Test:

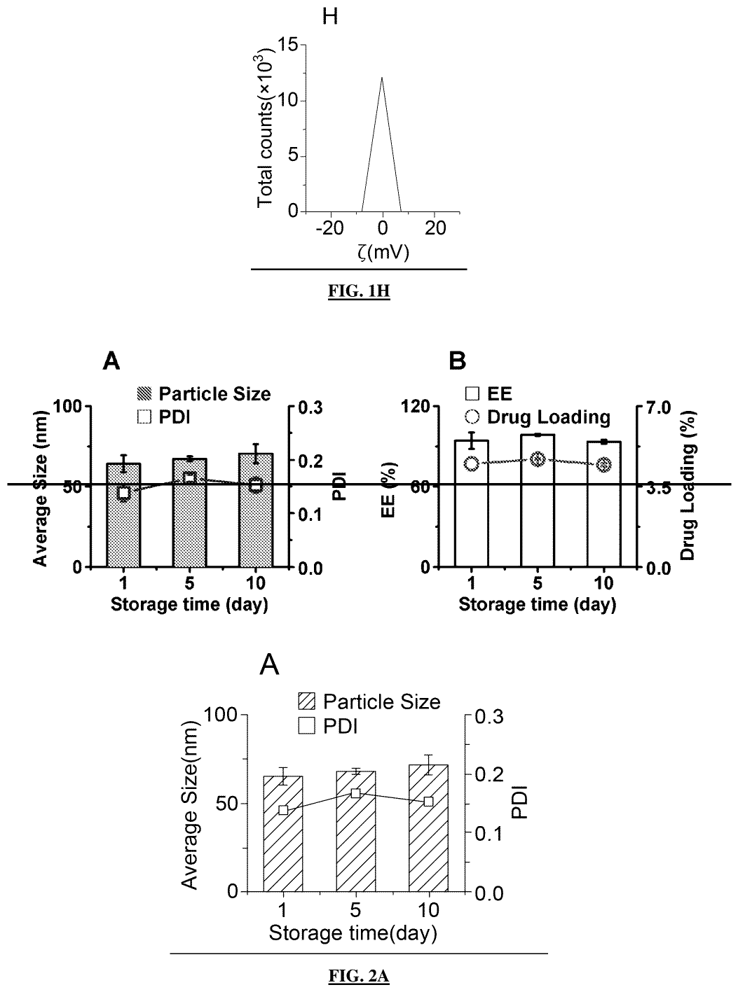

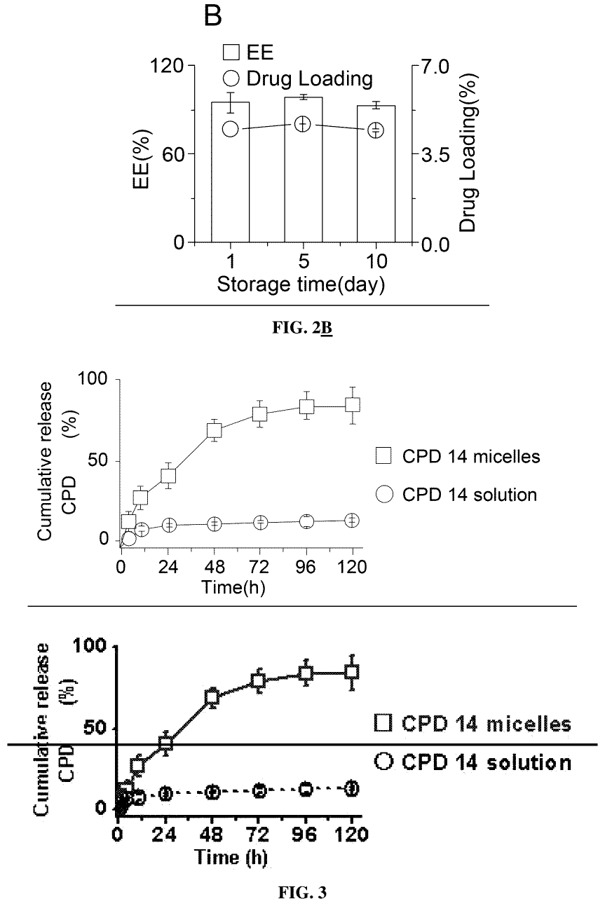

Taking the polymeric micelle prepared according to Example 1 as an example, samples were stored at 37° C., and sampling was conducted every 5 days to measure the average particle size, PDI, encapsulation efficiency, and drug loading capacity to evaluate the stability of the polymeric micelle. The results are shown in FIGS. 2A-2B. As shown in FIGS. 2A-2B, after the polymeric micelle of the present disclosure is stored for 10 days, the average particle size, PDI, encapsulation efficiency, and drug loading capacity all remain basically unchanged, indicating a high stability.

Cumulative Release Test:

Taking the polymeric micelle prepared according to Example 2 as an example, samples were placed in a PBS solution at 37° C. and pH=7.4. Sampling was conducted every day to quantitatively test a concentration of CPD14 molecules in the PBS solution using HPLC, while CPD14 molecules not entrapped by Soluplus were used as a control. The results are shown in FIG. 3. The results show that the polymeric micelle entrapped with drug has a complete drug release time that was significantly increased by at least 10 times compared with that of the un-entrapped drug, indicating that the drug could be released sustainably after being entrapped by the Soluplus carrier.

Stability test in blood:

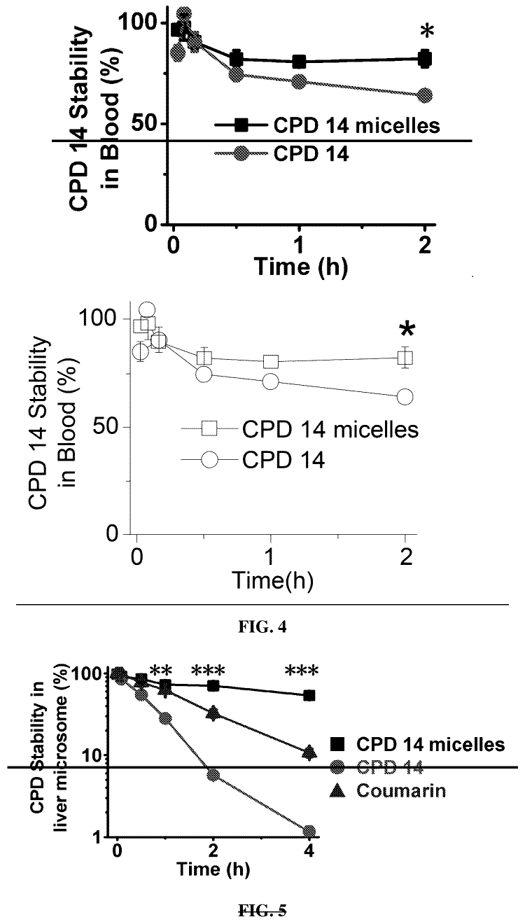

Taking the polymeric micelle prepared according to Example 2 as an example: in a 96-well plate, 10 μL of the polymeric micelle was added, then 190 μL of blood was added and shaken quickly to be uniform. At 37° C., a resulting mixture was taken out at preset time points of 5 min, 30 min, 60 min, and 120 min separately, and centrifuged at 3,000 rpm for 10 min to separate a plasma layer (upper layer) and a red blood cell layer (lower layer), each 100 μL. The plasma and red blood cell samples were treated using an organic solvent extraction method, and a concentration of CPD14 molecules in the samples was quantified and analyzed using HPLC (gradient method), while CPD14 molecules not entrapped by Soluplus were used as a control. The results are shown in FIG. 4.

The results showed that the drug-entrapped polymeric micelle still maintains a stability rate of 80% after being placed in the blood for 2 h, while the stability rate of drug not entrapped by Soluplus is significantly lower than that of the polymeric micelle. Therefore, the polymeric micelles prepared by using Soluplus to entrap drugs according to the present disclosure show a higher stability in blood, and do not cause the drug to decompose and become inactive in blood too quickly.

### Stability Test in Liver Microsomes:

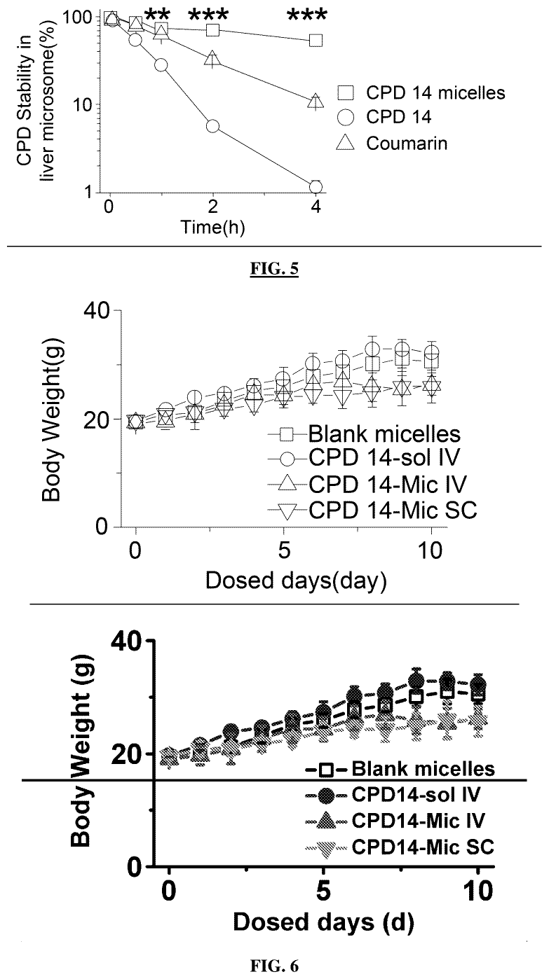

Taking the polymeric micelle prepared according to Example 2 as an example: a total volume of the human liver microsome experimental incubation system was 1,000 μL, including 500 μL of 1 mg/mL human liver microsome solution, 100 μL of the polymeric micelle solution of Example 2, 400 μL of NADPH coenzyme working solution (microsomes and coenzyme solution were both pre-incubated at 37° C. for 3 min to 5 min before adding). The system was incubated at a constant temperature of 37° C., and at the time points of the system incubation for 2 min, 5 min, 30 min, 60 min, 120 min, and 240 min, 200 μL of a reaction solution was taken, separately, and quickly added with 10 μL of 250 μg/mL internal standard Ebselen (DMSO), 8.5 μL of 1 M mercaptoethylamine, and 90 μL of 10 mM potassium dihydrogen phosphate (pH=7.5) in sequence, mixed to be uniform by vortexing, and placed in water bath at 37° C. for 3 h. After taking out the mixture, 600 μL of ethyl acetate was added, mixed to be uniform by vortexing, stood, and centrifuged at 10,000 rpm for 5 min to separate an ethyl acetate layer (upper layer). The upper layer was dried by blowing nitrogen, redissolved with 100 μL of methanol, centrifuged at 3,000 rpm for 2 min to obtain a supernatant, and the supernatant was subjected to HPLC analysis. The liquid chromatograms at relative retention time of Ebselen-BME and B series drug-BME were collected and quantitatively analyzed, while CPD14 molecules that were not entrapped by Soluplus were used as a control. The results are shown in FIG. 5. As shown in FIG. 5, the stability of the polymeric micelle in liver microsomes is significantly higher than that of un-entrapped drug. This indicates that Soluplus-entrapped drugs could reduce the metabolism of drugs in liver microsomes, to ensure that the drugs were not prematurely metabolized and inactivated before reaching the tumor sites.

### Cancer Cell Inhibition Test:

Taking the polymeric micelle prepared according to Example 2 as an example: the polymeric micelle was added to a 96-well plate, and 1,000 each of mouse lung cancer A549 cells and mouse liver cancer H22 cells were added to each well. 10% bovine serum was added to the Roswell Park Memorial Institute (RPMI) medium, and the cells were cultured in the RPMI medium under 5% CO₂ for 5 days, and 1 mL of erythrosin-methylene blue tetrazolium salt (EZMTT) indicator was added thereto to detect IC₅₀, while a Soluplus blank micelle without drug entrapment and CPD14 molecules not entrapped by Soluplus were used as a control. The results are shown in Table 5.

TABLE 5
Inhibition of H22 and A549 cancer cells by polymeric micelle

| Sample | H22 (IC₅₀: μM) | A549 (IC₅₀: μM) |
|---|---|---|
| CPD14 | 0.51 ± 0.04 | 0.49 ± 0.07 |
| Polymeric micelle | 075 ± 0.02 | 0.69 ± 0.18 |
| Soluplus blank micelle | >30 | >30 |

As shown in Table 5, the inhibitory effect of the polymeric micelle according to the present disclosure on H22 and A549 cancer cells is equivalent to that of a simple drug solution that was not entrapped by Soluplus, indicating that the entrapment with Soluplus carrier does not affect the activity of drugs.

### Bioavailability Test:

36 Institute of Cancer Research (ICR) mice weighing about 20 g were randomly divided into 2 groups, with 18 mice in each group. The mice all were separately administered using a CPD14 injection without Soluplus entrapment prepared according to Example 2 and the polymeric micelle prepared according to Example 2 in the same concentration, at a dose of 10 mg/kg by tail vein injection. Blood was collected at 0.003 h, 0.17 h, 0.5 h, 1 h, 2 h, 4 h, 8 h, 12 h, and 24 h after administration, and a plasma concentration of CPD14 was measured after treating the whole blood or plasma. The results are shown in Table 6.

TABLE 6
Pharmacokinetic parameters of CPD14
and polymeric micelle in vivo

| Parameter | CPD14 | Polymeric micelle |
|---|---|---|
| Dose (mg/kg) | 10 | 10 |
| AUC_{0 h→24 h} (μg/mL*h) | 9.6 ± 1.1 | 48.4 ± 3.7 |
| Cₘₐₓ (μg/mL) | 6.6 ± 0.7 | 68.9 ± 7.2 |
| Tₘₐₓ (h) | 0.033 | 0.033 |
| T_{1/2β} (h) | 1.7 ± 0.01 | 16.4 ± 4.3 |
| CL (L/h/kg) | 1.02 ± 0.12 | 0.10 ± 0.04 |
| Fᵣ (%) | — | 504.2 ± 19.4 |

AUC_{0→24 h}: an area under the curve from 0 h to 24 h; Cₘₐₓ: a peak plasma concentration of the compound; Tₘₐₓ: a time to peak plasma concentration; t_{1/2β}: half-life; CL: a clearance rate of the compound in blood; F r : relative bioavailability.

As shown in Table 6, the drug (polymeric micelle) entrapped by Soluplus has a peak plasma concentration in mice significantly higher than that of un-entrapped drug (CPD14), and has a longer half-life and a lower clearance rate in blood. This indicates that the polymeric micelle prepared according to the present disclosure could prolong an effective time of the drug in vivo and does not cause inactivation of the drug by hydrolyzation and oxidization after entering an organism. The polymeric micelle has a relatively high bioavailability.

### Treatment Test on Subcutaneous Tumor Transplants in Mice:

30 ICR mice weighing about 20 g were randomly divided into 3 groups, with 10 mice in each group. Liver cancer H22 cells were inoculated subcutaneously into the right armpit of mice. The next day, mice in Group 1 were injected with CPD14 injection without Soluplus prepared according to Example 2 at 20 mg/kg through the tail vein; mice in Group 2 waere injected with the polymeric micelle prepared according to Example 2 at 20 mg/kg through the tail vein; and mice in Group 3 were injected with the Soluplus blank micelle without drug at 20 mg/kg through the tail vein. The mice were injected once a day for 10 consecutive days, and the body weight of the mice was recorded and the status of the mice was observed every day. On the 11th day, the tumors of the mice were removed and measured. The results are shown in Table 7 and FIG. 6.

TABLE 7
Tumor weight of subcutaneous tumor transplants
of H22 mouse liver cancer after treatment

| Treatment drug | Tumor weight of H22 tumor transplant (g) |
|---|---|
| Soluplus blank micelle | 2.34 ± 0.06 |
| CPD14 | 2.15 ± 0.05 |
| Polymeric micelle | 0.87 ± 0.02 |

The results show that the Soluplus blank micelle without drug entrapment has no therapeutic effect on tumors, while the drug-entrapped polymeric micelle could significantly reduce the tumor weight. Compared with the drug not entrapped by Soluplus (CPD14), after treatment with the drug-entrapped polymeric micelle, the tumor weight is significantly reduced and the weight of the mice is also lower than that of the other two groups. This indicates that the Soluplus-entrapped drug (polymeric micelle) according to the present disclosure has a better therapeutic effect on the tumors.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

1. A poorly soluble chain-like drug-entrapped polymeric micelle, comprising a poorly soluble chain-like drug and polyvinylcaprolactam-polyvinyl acetate-polyethylene glycol (Soluplus);
wherein the poorly soluble chain-like drug is a selenium-containing glutaminase inhibitor, and a mass ratio of the poorly soluble chain-like drug to the Soluplus is in a range of 1: (5-100).

2. The poorly soluble chain-like drug-entrapped polymeric micelle of claim 1, wherein the poorly soluble chain-like drug has a structure as represented by one selected from the group consisting of: 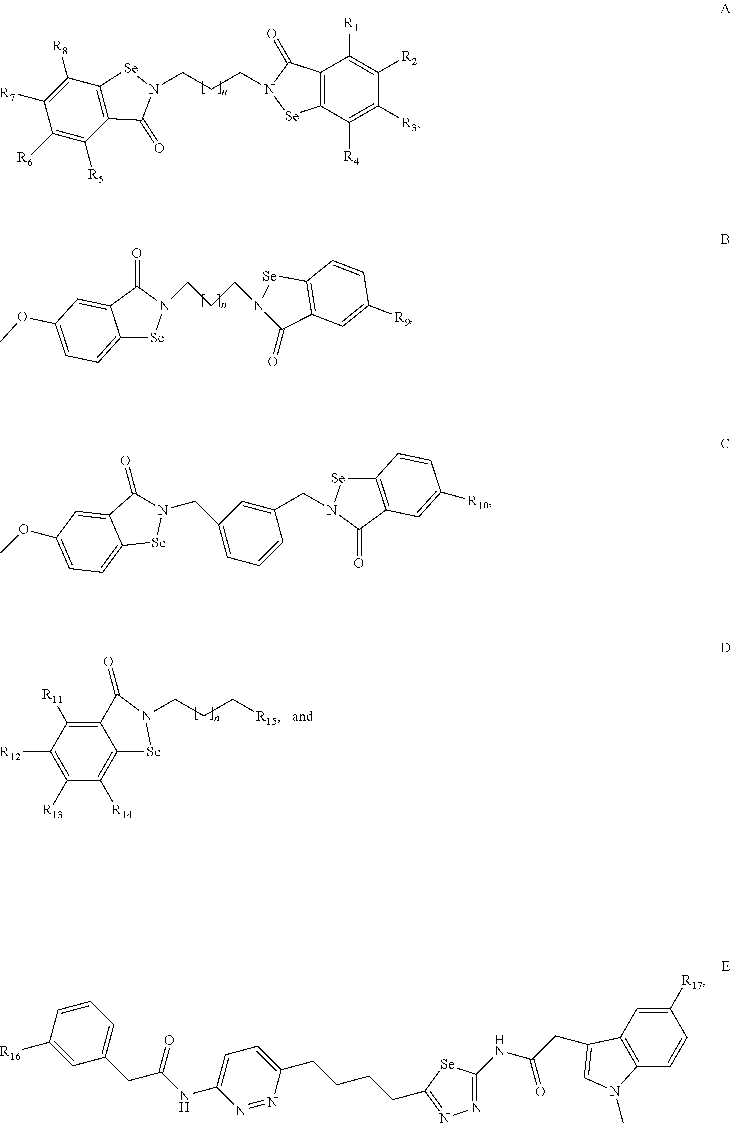
wherein, n is 1 to 4;
R₁ to R₈ are independently selected from the group consisting of H and F;
R₉ has a structure as represented by one selected from the group consisting of: 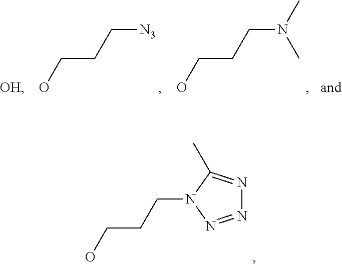
R₁₀ has a structure as represented by one selected from the group consisting of: 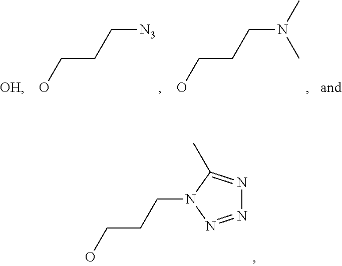
R₁₁ to R₁₄ are independently selected from the group consisting of H and F;
R₁₅ has a structure as represented by one selected from the group consisting of: 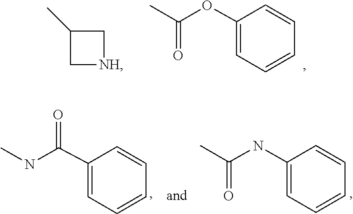
R₁₆ is selected from the group consisting of S and Se; and
R₁₇ is selected from the group consisting of H and OCH₃.

3. A method for preparing the poorly soluble chain-like drug-entrapped polymeric micelle of claim 1, comprising the following steps:
(1) dissolving the poorly soluble chain-like drug and the Soluplus in an organic solvent to obtain a mixed solution;
(2) subjecting the mixed solution obtained in step (1) to rotary evaporation to remove the organic solvent, to obtain a film, wherein the poorly soluble chain-like drug is entrapped with the Soluplus in the film; and
(3) subjecting the film obtained in step (2) to swelling hydration to obtain the poorly soluble chain-like drug-entrapped polymeric micelle.

4. The method of claim 3, wherein the Soluplus in step (1) has a molecular weight of 90,000 to 140,000.

5. The method of claim 3, wherein the organic solvent in step (1) is one or more selected from the group consisting of methanol, dichloromethane, ethanol, diethyl ether, and acetonitrile.

6. The method of claim 5, wherein in step (1), a ratio of a mass of the Soluplus to a volume of the organic solvent is in a range of (0.5-100) mg: 5 mL.

7. The method of claim 3, wherein a solution for the swelling hydration in step (3) is selected from the group consisting of deionized water, physiological saline, and phosphate-buffered saline; and a ratio of a volume of the solution for the swelling hydration to a mass of the Soluplus is in a range of (2-12) mL: 200 mg.

8. The method of claim 3, wherein the poorly soluble chain-like drug-entrapped polymeric micelle obtained in step (3) is subjected to a post-treatment to obtain a lyophilizer of the poorly soluble chain-like drug-entrapped polymeric micelle; and the post-treatment comprises: adding a lyoprotectant into the poorly soluble chain-like drug-entrapped polymeric micelle to obtain a mixture, and subjecting the mixture to filtration, sterilization, and freeze-drying in sequence to obtain the lyophilizer of the poorly soluble chain-like drug-entrapped polymeric micelle.

9. The method of claim 8, wherein the lyoprotectant is one or more selected from the group consisting of mannose, lactose, glucose, an amino acid, sucrose, and polyethylene glycol; and
the lyoprotectant is used in an amount of 1% to 5% of a mass of the poorly soluble chain-like drug-entrapped polymeric micelle.

10. An anti-tumor drug, an antibacterial drug, or an anti-inflammatory drug, comprising the poorly soluble chain-like drug-entrapped polymeric micelle of claim 1, wherein the anti-tumor drug, the antibacterial drug, or the anti-inflammatory drug.

11. The method of claim 3, wherein the poorly soluble chain-like drug has a structure as represented by one selected from the group consisting of: 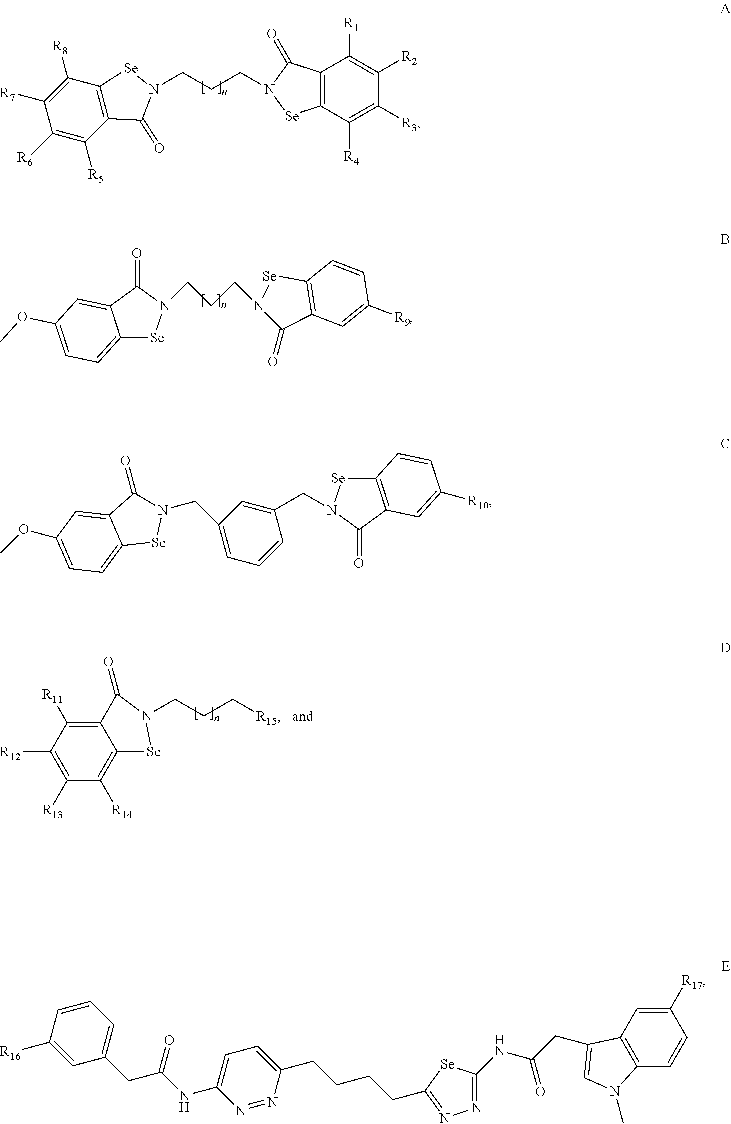
wherein, n is 1 to 4;
R₁ to R₈ are independently selected from the group consisting of H and F;
R₉ has a structure as represented by one selected from the group consisting of: 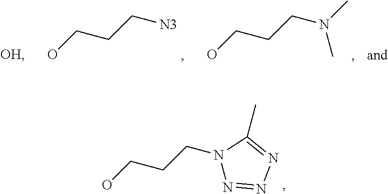
R₁₀ has a structure as represented by one selected from the group consisting of: 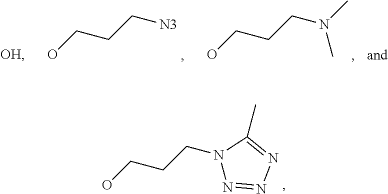
R₁₁ to R₁₄ are independently selected from the group consisting of H and F;
R₁₅ has a structure as represented by one selected from the group consisting of: 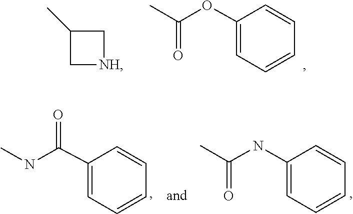
R₁₆ is selected from the group consisting of S and Se; and
R₁₇ is selected from the group consisting of H and OCH₃.

12. The method of claim 4, wherein the organic solvent in step (1) is one or more selected from the group consisting of methanol, dichloromethane, ethanol, diethyl ether, and acetonitrile.

13. The anti-tumor drug, the antibacterial drug, or the anti-inflammatory drug of claim 10, wherein the poorly soluble chain-like drug has a structure as represented by one selected from the group consisting of: 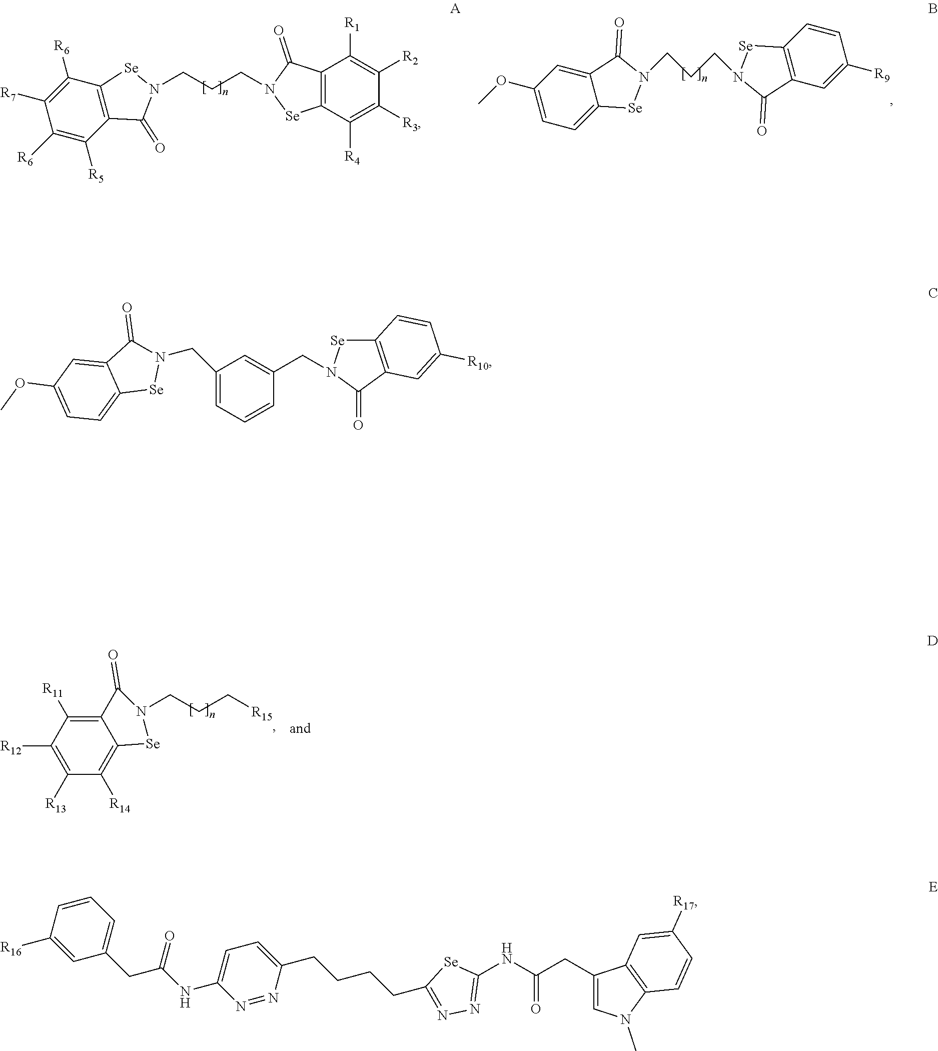
wherein, n is 1 to 4;
R₁ to R₈ are independently selected from the group consisting of H and F;
R9 has a structure as represented by one selected from the group consisting of: 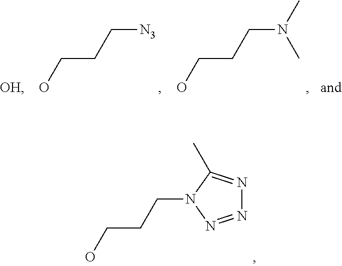
R₁₀ has a structure as represented by one selected from the group consisting of: 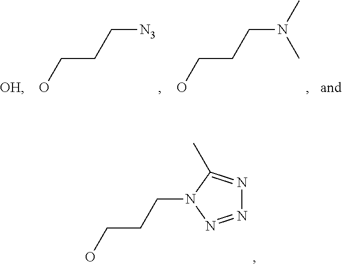
R₁₁ to R₁₄ are independently selected from the group consisting of H and F;
R₁₅ has a structure as represented by one selected from the group consisting of: 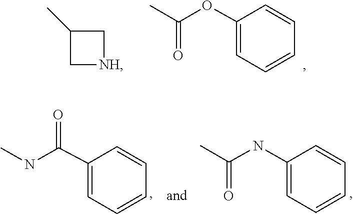
R₁₆ is selected from the group consisting of S and Se; and
R₁₇ is selected from the group consisting of H and OCH₃.
